(19) United States
(12) Patent Application Publication
(10) Pub. No.: US 2006/0122147 A1
(43) Pub. Date: Jun. 8, 2006

(54) Combination preparation of hyaluronic acid and at least oe local anesthetic and the use thereof

(76) Inventor: David Wohlrab, Halle (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/529,924

(22) PCT Filed: Sep. 9, 2003
(86) PCT No.: PCT/EP03/10822

§ 371 Date: Nov. 28, 2005

(30) Foreign Application Priority Data
Oct. 4, 2002 (DE) 102 46 340.9

(51) Int. Cl. A61K 31/728; C08B 37/00

(52) U.S. Cl. 514/54; 536/53

(57) ABSTRACT

The invention relates to a combination preparation comprising an active agent A from the group hyaluronic acid, the salts and fragments thereof, at least one active agent B from the group of local anaesthetics and derivatives thereof and also if necessary further additives. These combination preparations are used for the medical treatment of degenerative and traumatic diseases of all joints, for the treatment of articular cartilage and cartilage bone defects and also meniscus and intervertebral disc lesions, such as e.g. arthrosis, articular rheumatism, osteochondritis dissecans, flake fractures, meniscus lesions and for the treatment of skin and mucous membrane changes, also from cosmetic aspects.

The invention relates to a combination preparation comprising an active agent A from the group hyaluronic acid, the salts and fragments thereof, at least one active substance B from the group of local anaesthetics and derivatives thereof and also if necessary further additives. These combination preparations are used for the medical treatment of degenerative and traumatic diseases of all joints, for the treatment of articular cartilage and cartilage bone defects and also meniscus and intervertebral disc lesions, such as e.g. arthrosis, articular rheumatism, osteochondritis dissecans, flake fractures, meniscus lesions and for the treatment of skin and mucous membrane changes, also from cosmetic aspects.

The chemical name for hyaluronic acid is hyaluronan. Its chemical structure corresponds to the formula

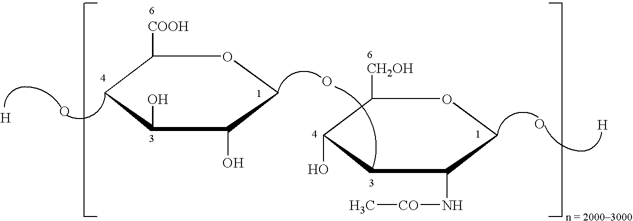

Despite the positive clinical experiences with high molecular hyaluronic acid and the salts thereof (mol mass >1×106⁶ Dalton), knowledge about the operating mechanism is incomplete. The present state of knowledge identifies intraarticularly applied hyaluronic acid as a lubricant (A. Lussier et al. (1996); J. Rheumatol. 23, 1579-1585; D. Scale et al. (1994); Current Therapeutic Research. 55, 220-232; M. Wobig et al. (1998) Clinical Therapeutics. 20, 410-423). Furthermore, it was indicated that hyaluronic acid has intraarticularly anti-inflammatory properties (K. W. Marshall (1997) Today's Therapeutic Trends. 15, 99-108; K. W. Marshall (2000) Curr. Opin. Rheumatol. 12, 468-474).

Arthrosis begins with initial damage of the cartilage tissue because of various causes. This results in reactive synovialitis which for its part causes both pathological changes in the synovial fluid, i.e. reduction in concentration and molecular weight of the hyaluronic acid, and also the release of inflammation mediators. This leads to secondary cartilage damage and hence finally to arthrosis which, in addition to cartilage tissue, affects all other articular structures (J. P. Pelletier et al. (1993) J. Rheumatol. 20, 19-24).

It is known that intraarticularly applied hyaluronic acid leads to improvement in joint mobility, to pain reduction, to inhibition of inflammation processes and, under in vitro conditions, to the increase of chondrocyte proliferation (K. Kawasaki et al. (1999) Cell Physiol. 179, 142-148; D. Wohlrab et al. (2000) hylan news. 2; 2-5).

Starting herefrom, it was the object of the present invention to provide a combination preparation which can be applied in various forms and in the case of which the active substances can be released in a specifically delayed manner.

This object is achieved by the combination preparation with the features of claim 1. The use of the combination preparation is described in claim 15. The further dependent claims demonstrate advantageous developments.

According to the invention, a combination preparation consisting of an active agent A from the group hyaluronic acid, the physiological salts and fragments thereof, at least one active agent B from the group of local anaesthetics and derivatives thereof and also if necessary further additives is provided.

It was established that, due to the considerable molecular size of hyaluronic acid (1-6×10⁶ Da), this must be split several times before it can leave the intraarticular space and be decomposed or incorporated in cartilage tissue. These splitting processes take hours up to several days dependent upon the mol mass of the hyaluronic acid.

Because of this extended intraarticular dwell time, in comparison to other low molecular substances, such as e.g. local anaesthetics, high molecular hyaluronic acid, the salts or the fragments thereof are suitable as carriers for substances which, without bonding of this type to a carrier molecule, have a significantly shortened intraarticular dwell time and hence a very short period of activity.

All the formulations known from the state of the art are possible as galenic formulation. Included herein are in particular intraarticularly, intradiscally, subcutaneously, intracutaneously or topically applicable galenic formulations.

Preferably, compounds chosen as active agent A are compounds from the group hyaluronic acid, the salts and fragments thereof and, as active agent B, compounds from the group of local anaesthetics and derivatives thereof, which compounds have together a chemical or physical bond, the active substance B being able to be released in a delayed manner. The pH value of the formulation thereby makes possible an optimum bond between the two active agents and the release of the active agent B can be controlled via alteration in the pH value of the surrounding medium.

Preferably, the active agent A is contained in the combination preparation in a concentration between 0.001 and 5% by weight or preferably between 0.2 and 2.0% by weight. The active agent B is preferably in a concentration between 0.001 and 20% by weight, preferably between 0.001 and 5.0% by weight.

Furthermore, further additives can be contained in the combination preparation. There are included herein for example agents with radical interceptor properties, in particular tocopherol derivatives or ascorbic acid derivatives. Furthermore, agents of the hyaline cartilage tissue can be used, in particular glucosamine sulphate derivatives or chondroitin sulphate derivatives. Furthermore, agents with a steroidal and corticoidal effect can be used, in particular glucocorticoids. There are possible as additives furthermore non-steroidal antiphlogistics which are described also as antirheumatics, in particular indometacin, diclofenac or salicylic acid derivatives and analgesics, in particular oxicams, aniline or anthranilic acid derivatives. The combination preparation can have as additive likewise substances with an inhibitory effect on prostaglandin synthesis, in particular lipoxygenase inhibitors, cyclo-oxygenase inhibitors and phospholipase A2 inhibitors. Likewise, there are possible as additives growth factors, in particulars retinol or bone morphogenetic proteins (BMPs), vitamins, in particular vitamin A, C, B12 or biotin, antioxidants, in particular flavonoids or glutathione, and agents with water-binding properties, in particular urea or arginine.

The combination preparation can be produced as any galenic formulation, e.g. as a solution, suspension, emulsion, paste, ointment, gel, cream, lotion, varnish, powder, soap, surfactant-containing cleaning preparation, oil, lipstick, lip salve, mascara, eye liner, eye shadow, rouge, powder, emulsion or wax makeup, sun protection, pre-sun and after-sun preparations or as a spray.

The application of the combination preparation can be effected both on humans and on animals. The combination preparations according to the invention can be applied both in human and veterinary medicine and in cosmetics.

The application fields of the combination preparations relate to human and veterinary medical therapy, prophylaxis and/or metaphylaxis of degenerative or traumatic articular diseases and articular function disorders, articular cartilage and cartilage bone defects, meniscus and intervertebral disc diseases. There are included herein for example the increase in chondrocyte proliferation, the stabilisation and/or regeneration of articular structures, in particular of the articular cartilage and menisci, the increase in joint mobility and the inhibition of inflammatory processes.

Likewise, the combination preparation can however by used also for treating skin and mucous membrane changes both from medical and cosmetic viewpoints.

According to the invention, also the use of at least one active agent from the group hyaluronic acid, the salts and fragments thereof in combination with at least one active agent B from the group of local anaesthetics and derivatives thereof for preparing a medicament for human and veterinary medical therapy, prophylaxis and/or metaphylaxis of articular diseases and articular function disorders is provided.

The invention is intended to be explained with reference to the following Examples and Figures without restricting it thereto.

### EXAMPLE 1

### Physiological Compatibility of the Galenic Formulations According to the Invention

Production:

Lidocaine hydrochloride (University pharmacy of the Martin Luther University Halle-Wittenberg) and hyaluronic acid (Aqua Biochem, Dessau) (MG 1.5×10⁶ Da) were present primarily in powder form. In order to produce 2% parent solutions, corresponding quantities were dissolved in RPMI medium (Seromed, Berlin) and subsequently filtered in a sterile manner. In order to produce a lidocaine-hyaluronic acid mixture, these parent solutions were mixed in equal parts. The substance addition to the cell culture was effected on the 10^{th} culture day with medium change. Corresponding quantities of the test substances (parent solutions) were added here so that a respective end concentration of 5×10⁻⁵ mmol/1 was achieved.

Preparation of the Biological Material:

The tests were effected on human chondrocytes which were isolated from arthrotically changed knee joint cartilage. The cartilage tissue stemmed from femoral articular surfaces resected during implantation of total knee endoprostheses. Exclusively arthrotically changed cartilage tissue from three different donors without known relevant secondary diseases, in particular without rheumatoid arthritis, was used.

The intraoperatively obtained bone-cartilage fragments were transferred firstly into sterile L15 medium (Seromed, Berlin) as transport medium. Subsequently, the separation of the cartilage tissue from the subchondral bone was effected under sterile conditions by means of a scalpel and also sharp severance of the tissue into pieces of approximately 1 mm³. The enzymatic isolation of the chondrocytes from the pieces of cartilage was effected by means of pronase and collagenase A (Boehringer Mannheim) over a timespan of 16 hours.

Test Conditions:

The isolated chondrocytes were cultivated in 24 well plates in RPMI medium (Seromed, Berlin) with the addition of various antibiotics at 37° C. and 5% carbon dioxide in an incubator as a monolayer culture. The medium change was effected every 2 days. After 10 culture days, finally a medium change was effected and hereby the addition of the respective test substances which were dissolved in the culture medium. In addition, an untreated chondrocyte population respectively was run jointly as control.

Implementation of the Test:

The measurement of the ³H thymidine incorporation as a measure of the DNA synthesis yield was effected 24, 48 or 72 hours after addition of the substance. At the end of the culture time, 20 μl ³H-methylthymidine (specific activity 60.3 Ci/mmol; American Radiolabeled Chemicals Inc., St. Louis, USA) was added per well to the cell culture. Two hours after the addition of the ³H thymidine, the medium was suctioned out of the chambers by means of a Cell Harvester (Berthold GmbH, Bad Wildbad). Each culture chamber was supplied with 200 μl trypsine and the cell suspension was suctioned off after 20 minutes via a filter. Subsequently, the measurement of the radioactivity of the cells in the filter paper was effected by means of a liquid scintillation meter (WINSPECTRAL 1414, Wallace-ADL GmbH, Freiburg, Deutschland).

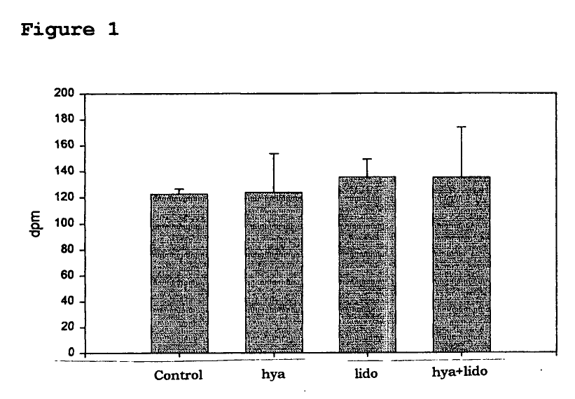

The results for the physiological compatibility of the galenic formulations according to the invention are represented in FIG. 1.

FIG. 1 shows the effect of hyaluronic acid (hya) (1.5×10⁶ Da, 5×10⁻⁵ mmol/l), lidocaine (lido) (5×10⁻⁵ mmol/l) and hyaluronic acid-lidocaine mixture (hya +lido) (respectively 5×10⁻⁵ mmol/l) on the incorporation of ³H thymidine by human chondrocytes (N=3) cultivated in vitro after 48 h. Each measurement value is the mean of 8 individual measurements.

### EXAMPLE 2

### Influence of Lidocaine Upon the Proliferation of Human Chondrocytes

Production:

Lidocaine hydrochloride (University pharmacy of the Martin Luther University Halle-Wittenberg) was present primarily in powder form. This was dissolved in a corresponding quantity in RPMI medium (Seromed, Berlin) so that an end concentration of 0.1 mmol/l lidocaine was present. Sterile filtration was effected subsequently. The addition of substance to the cell culture was effected after the 2^{nd} culture day during each medium change. Corresponding quantities of the test substances (parent solutions) were added here so that a respective end concentration of 5×10⁻⁵ mmol/l was achieved.

Preparation of the Biological Material:

The preparation of the cartilage tissue and the chondrocytes isolated therefrom was effected analogously to the methods represented in Example 1.

Test Conditions:

The isolated chondrocytes were cultivated in 24 well plates in RPMI medium (Seromed, Berlin) with the addition of various antibiotics at 37° C. and 5% carbon dioxide in an incubator as a monolayer culture. The medium change was effected every two days. After the first medium change, the addition of lidocaine in the cell culture medium was effected in a concentration of 0.1 mmol/l. In addition, an untreated chondrocyte population respectively was run jointly as control. The culture duration was 6, 12 or 18 days.

Implementation of the Test:

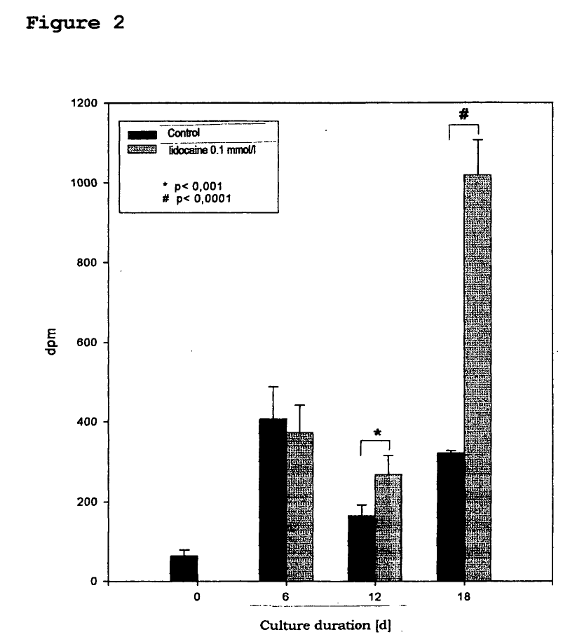

The measurement of the ³H thymidine incorporation as a measure of the DNA synthesis yield was effected at the respective end of the culture duration analogously to the method represented in Example 1. The results of the tests are represented in FIG. 2.

FIG. 2 shows the effect of lidocaine (0.1 mmol/l) on the ³H thymidine incorporation by human chondrocytes (N=6) cultivated in vitro. Substance addition on the second culture day. Each measurement value is the mean of 8 individual measurements.

### EXAMPLE 3

### The Optimal Bonding of the Local Anaesthetic or Local Anaesthetics or the Derivatives of These Compounds to Hyaluronic Acid and/or the Physiologically Compatible Salts of Hyaluronic Acid and also the Fragments of these Compounds in the Example of Lidocaine

TABLE 1
shows subsequently the proportion of free lidocaine with
different lidocaine concentrations (conc. hyaluronic acid = 0.05%).

| | | Conc. lidocaine [%] | | | | | | |
|---|---|---|---|---|---|---|---|---|
| | | 0.025 | 0.05 | 0.01 | 0.2 | 0.3 | 0.4 | 0.5 |
| Free | pH 6.9 | 54 | 55 | 70 | 71 | 87 | 89 | 93 |
| lidocaine [%] | pH 7.9 | 34 | 81 | 79 | 82 | 89 | 91 | 105 |

Experimental Conditions:

3D CE system of the company Hewlett Packard with fused silica capillary 40.0 (48.5) cm with internal diameter 50 μm, temp.: 25° C., pressure injection: 50 mbar×sec, voltage: +30 kV, UV detection: cathodeside at λ=195 nm and 200 nm, injection time: 200 sec. Throughout the injection time, 7.5 cm of the capillary was filled with the sample in order to achieve optimal separation of the peaks.

By means of electrophoretic frontal analysis, it was able to be shown that an interaction took place between hyaluronic acid (hya) and lidocaine. If the same percentage proportions of hya and lidocaine or if percentage-wise less lidocaine than hya is present, the greatest proportion of lidocaine is bonded to hya. The mechanism of the interaction is based on incorporation of the lidocaine in the helix-like coil of the hya, there also at pH value of 7.9, when lidocaine is present semi-undissociated (pKs value=7.9 in the presence of hya). In addition, ionic bonds are involved in the interaction, since at pH 6.9 when lidocaine is present completely dissociated, less free lidocaine was able to be detected (except with a lidocaine concentration=0.025%)

### EXAMPLE 4

### Delayed Release of the Local Anaesthetic or Local Anaesthetics or the Derivatives of this Compound From Formulations Which Contain Hyaluronic Acid and/or the Physiologically Compatible Salts of Hyaluronic Acid and of Fragments of These Compounds, in the Example of Lidocaine

TABLE 2
shows subsequently the flux of lidocaine through a dialysis membrane
with and without hyaluronic acid (hya) in the donor compartment.

| | | pH value | | | | | |
|---|---|---|---|---|---|---|---|
| | | 3.1 | 6.0 | 6.5 | 6.9 | 7.7 | 9.0 |
| Flux | lidocaine | 0.32 | 0.355 | 0.315 | 0.42 | 0.35 | 0.09 |
| [mg h⁻¹ | lidocaine + | 0.27 | 0.256 | 0.234 | 0.27 | 0.23 | 0.04 |
| cm⁻²] | hya | | | | | | |

| | | | | | | |
|---|---|---|---|---|---|---|
| Difference [%] | | | | | | |
| Flux _{lidocaine} − | 15.6 | 27.8 | 25.8 | 35.7 | 34.3 | 55.6 |
| Flux _{lidocaine + hya} | | | | | | |

Experimental Conditions:

diffusion cell with diffusion surface (A)=15.9 cm³ and a sodium cellulose xanthate (nephrophan) dialysis membrane, volume (V) of the donor (DK) and of the acceptor compartment (AK)=20 ml, diffusion time=4 h, temp.: 37° C., concentration of the hya in the donor compartment=0.25% and the initial concentration of the lidocaine in the donor compartment=0.05%.
Calculation of the Flux:

$$Flux = \frac{{C}_{AK} {V}_{AK}}{At} .$$

Thereby:
C_{AK}=Concentration of the lidocaine in the AK and
t=diffusion time.

With reference to the results which were obtained in the dialysis cell, it was shown that the flux of the lidocaine through this pore membrane was reduced considerably in the presence of the hya in the donor compartment. The most pronounced is the effect at pH=9.0, there lidocaine is present extensively undissociated. This confirms the results which were described in Example 3 that the mechanism of the interaction between lidocaine and hya is based on incorporation of the lidocaine in the helix-like coil of the hya. But also at pH values between 6.9 and 7.7, a strong reduction in the lidocaine flux can be observed. This confirms that also ionic bonds and the interaction between lidocaine and hya are involved.

If the pH value is moved into the acidic range, e.g. after pH=3.1, the hya there is present extensively undissociated, the lidocaine flux is reduced less strongly. This shows clearly that also ionic bonds are involved in the interaction.

It can be established in total that a strong delay effect with respect to the release of the lidocaine from the lidocaine-hya complex can be achieved. As a result, the effect of the lidocaine in biological systems (e.g. in the knee joint) can be considerably extended.

1. Combination preparation consisting of at least one active agent A from the group hyaluronic acid, the salts and fragments thereof, an active agent B from the group of local anaesthetics and derivatives thereof and if necessary further additives for human and veterinary medical therapy, prophylaxis and/or metaphylaxis of degenerative or traumatic articular diseases and articular function disorders and also articular cartilage and cartilage bone defects.

2. Combination preparation according to claim 1, wherein the active agents A and B are bonded to each other chemically or physically, and in that the active agent B can be released in a delayed manner.

3. Combination preparation according to claim 1, wherein the active agent A is contained in a concentration between 0.001 and 5% by weight, preferably between 0.2 and 2% by weight.

4. Combination preparation according to claim 1, wherein the active agent B is contained in a concentration between 0.001 and 20% by weight, preferably between 0.001 and 5% by weight.

5. Combination preparation according to claim 1, wherein agents with radical interceptor properties, in particular tocopherol derivatives and/or ascorbic acid derivatives, are contained as additive.

6. Combination preparation according to claim 1, wherein agents of the hyaline cartilage tissue, in particular glucosamine sulphate derivatives and/or chondroitin sulphate derivatives, are contained as additive.

7. Combination preparation according to claim 1, wherein agents with a steroidal or corticoidal effect, in particular glucocorticoids, are contained as additive.

8. Combination preparation according to claim 1, wherein non-steroidal antiphlogistics, in particular indometacin, diclofenac or salicylic acid derivatives, are contained as additive.

9. Combination preparation according to claim 1, wherein analgesics, in particular oxicams, aniline or anthranilic acid derivatives, are contained as additive.

10. Combination preparation according to claim 1, wherein agents with an inhibitory effect on prostaglandin synthesis, in particular lipoxygenase inhibitors, cyclo-oxygenase inhibitors and phospholipase A2 inhibitors, are contained as additive.

11. Combination preparation according to claim 1, wherein growth factors, in particular retinol or bone morphogenetic proteins (BMPs), are contained as additive.

12. Combination preparation according to claim 1, wherein vitamins, in particular vitamin A, C, B12 or biotin, are contained as additive.

13. Combination preparation according to claim 1, wherein antioxidants, in particular flavonoids or glutathione, are contained as additive.

14. Combination preparation according to claim 1, wherein agents with water-binding properties, in particular urea or arginine, are contained as additive.

15. Use of hyaluronic acid, the salts and fragments thereof in combination with at least one active agent from the group of local anaesthetics and derivatives thereof for preparing a medicament for human and veterinary medical therapy, prophylaxis and/or metaphylaxis of degenerative or traumatic articular diseases and articular function disorders.

16. Use according to claim 15 for human and veterinary medical therapy, prophylaxis and/or metaphylaxis of articular cartilage and cartilage bone defects.

17. Use according to claim 15 for human and veterinary medical therapy, prophylaxis and/or metaphylaxis of meniscus and intervertebral disc diseases.

18. Use according to claim 15 in order to increase the proliferation of chondrocytes.

19. Use according to claim 15 in order to stabilise and/or regenerate articular structures, in particular of the articular cartilage and of the meniscus.

20. Use according to claim 15 in order to increase articular mobility.

21. Use according to claim 15 in order to inhibit inflammatory processes.

22. Use according to claim 15 in order to treat skin or mucous membrane changes.
